(19) United States
(12) Patent Application Publication
(10) Pub. No.: US 2008/0241292 A1
(43) Pub. Date: Oct. 2, 2008

(54) COMPOSITION AND METHOD FOR WEIGHT LOSS

(75) Inventors: Marvin A. Heuer, Mississauga (CA); Ken Clement, Mississauga (CA); Shan Chaudhuri, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: IOMEDIX DEVELOPMENT INTERNATIONAL SRL, St. Michael (BB)

(21) Appl. No.: 11/867,499

(22) Filed: Oct. 4, 2007

Related U.S. Application Data
(60) Provisional application No. 60/863,222, filed on Oct. 27, 2006.

(51) Int. Cl. A61K 36/605

(52) U.S. Cl. 424/774

(57) ABSTRACT

A nutritional composition for promoting weight loss by jointly and simultaneously suppressing appetite, increasing satiation, decreasing the metabolic breakdown of carbohydrates and decreasing the absorption of carbohydrates in an individual comprising, effective amounts of an extract of Mulberry leaf, pinolenic acid and an extract of Salacia oblonga is provided. Both a composition and a method are provided by the present disclosure.

### RELATED APPLICATIONS

The present application is related to and claims benefit of priority to U.S. Provisional Application No. 60/863,222 entitled “Composition and method for weight loss” filed Oct. 27, 2006, the disclosure of which is hereby fully incorporated by reference.

### FIELD OF THE INVENTION

The present invention is related to nutritional compositions for promoting weight loss in an individual. More specifically, the present invention relates to a nutritional composition comprising a combination of an extract of Mulberry leaf, a source of pinolenic acid and an extract of Salacia oblonga.

### BACKGROUND OF THE INVENTION

Ingested food is broken down mechanically and chemically in the gastrointestinal tract for use in the body. For example, carbohydrates are digested in the stomach and intestine to monosaccharides such as glucose, lipids are digested to fatty acids and monoacylglycerols, and proteins are digested to amino acids. These digested components along with other nutrients such as vitamins and minerals are absorbed by the body. The process involves numerous coordinated events including hormonal and chemical signaling.

A particularly important signaling group is that responsible for satiety, or the sensation of being full to reduce or cease food intake to prevent overeating is Cholecystokinin (CCK); the most widely studied satiety signal peptide. CCK is secreted from intestinal cells in response to nutrients. Administration of exogenous CCK to humans causes a reduction in food intake (Woods S C. Gastrointestinal satiety signals 1. An overview of gastrointestinal signals that influence food intake. Am J Physiol Gastrointest Liver Physiol. 2004 January; 286(1):G7-13). Glucagon-like peptide-1 (GLP-1) is another important satiety signal peptide. Like CCK, GLP-1 has been shown to increase satiety in humans (Naslund E, Gutniak M, Skogar S, Rossner S, Hellstrom P M. Glucagon-like peptide 1 increases the period of postprandial satiety and slows gastric emptying in obese men. Am J Clin Nutr. 1998 September; 68(3):525-30).

Alpha-glucosidase (α-glucosidase) is an enzyme that catalyzes the hydrolysis of maltose to glucose and is thus important for metabolizing more complex carbohydrates into simple sugars which can then be absorbed through the intestine for use with the cells of the body. Inhibitors of α-glucosidase have shown therapeutic efficacy for reducing carbohydrate absorption and postprandial glucose increases in humans (Brewer D. Are alpha-glucosidase inhibitors effective for control of type 2 diabetes? Am Fam Physician. 2006 Feb. 1; 73(3):433-4).

Weight management may be significantly aided by concomitantly increasing the satiety and decreasing the absorption of carbohydrates in an individual.

### SUMMARY OF THE INVENTION

The present invention is directed towards a nutritional composition comprising an effective amount of an extract of Mulberry leaf, a source of an effective amount of pinolenic acid and an effective amount of an extract of Salacia oblonga. The ingredients of the present composition act substantially simultaneously to suppress appetite, increase satiation, decrease the metabolic breakdown of carbohydrates and decrease the absorption of carbohydrates in said mammal. Both a composition and a method are provided by the present disclosure.

In various embodiments, the method and composition may comprise multi-phasic dissolution characteristic of the ingredients, providing time-release mechanisms.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional composition for promoting weight loss in an individual by acting to concomitantly increase the satiety of an individual by increasing CCK and GLP-1 activity, and decreasing the absorption of carbohydrates by inhibiting the activity of a-glucosidase.

It is herein understood that the activity of signaling molecules such as CCK and GLP-1 are the result of the activity of mature signaling molecules in concert with the corresponding receptors. As such, the activity of such signaling molecules may be modulated by affecting any number of mechanisms including but not limited to: signaling molecules and receptor synthesis, secretion, modification, transport, and recognition and binding.

It is also understood that the activity of enzymes such as a-glucosidase are the result of the activity of the mature protein. As such, the activity of such enzymes may be modulated by affecting any number of mechanisms including but not limited to: enzyme transcription, translation, post-translational modification, secretion, transport and cofactor binding.

It is further understood that the body weight of an individual is determined by the effect of both caloric intake from ingested absorbed food and caloric expenditure from basal metabolism and activity. As such reducing the calories absorbed by reducing food intake due to feelings of ‘fullness’ due to increased satiety and reduced carbohydrate absorption will have the net effect of promoting weight loss.

As used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’ as disclosed herein belong to category of compositions having at least one physiological function when administered to a mammal by conventional routes of administration.

Alternatively, formulations and nutritional compositions belonging to the present invention may be considered to be nutraceuticals. As used herein, the term ‘nutraceutical’ is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer any substance that is a food, a part of food, or an extract of food which is suitable for consumption by an individual and providing physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, Soft-Gel™ caplets, gel-caps and the like, not associated with food.

Extracts suitable for use in the present invention may be produced by extraction methods as are known and accepted in the art such as alcoholic extraction, aqueous extractions, carbon dioxide extractions, for example.

Mulberry

Mulberry (Morus alba) is an edible plant used in Chinese medicine rich in flavonoids with antioxidant activity (Doi K, Kojima T, Makino M, Kimura Y, Fujimoto Y. Studies on the constituents of the leaves of Morus alba L. Chem Pharm Bull (Tokyo). 2001 February; 49(2):151-3). Mulberry leaves have been shown to result in weight loss and reduced postprandial glucose increase, indicative of reduced carbohydrate absorption (Enkhmaa B, Shiwaku K, Katsube T, Kitajima K, Anuurad E, Yamasaki M, Yamane Y. Mulberry (Morus alba L.) leaves and their major flavonol quercetin 3-(6-malonylglucoside) attenuate atherosclerotic lesion development in LDL receptor-deficient mice. J. Nutr. 2005 April; 135(4):729-34). Furthermore, a tea extract containing Mulberry has been shown to reduce carbohydrate absorption in humans (Zhong L, Furne J K, Levitt M D. An extract of black, green, and mulberry teas causes malabsorption of carbohydrate but not of triacylglycerol in healthy volunteers. Am J Clin Nutr. 2006 September; 84(3):551-5) which is thought to be due to constituents of Mulberry that inhibit a-glucosidase.

It is herein understood by the inventors that the incorporation of an extract of Mulberry in a nutritional composition for promoting weight loss will effectively inhibit the absorption of carbohydrates from ingested food by inhibiting α-glucosidase.

An embodiment of the present invention comprises between from about 0.01 g to about 2.0 g of an extract of Mulberry leaves per serving of the supplemental composition. In an embodiment, the supplemental composition comprises from about 0.05 g to about 1.5 g of an Extract of Mulberry leaves per serving of supplement composition. In a further embodiment, the supplemental composition comprises about 1.2 g of an Extract of Mulberry leaves per serving of supplemental composition.

Pinolenic Acid

Pinolenic acid is a triple-unsaturated fatty acid which is a positional isomer of the more common gamma-linolenic acid which is found exclusively in pine nut oil. At a meeting of The American Chemical Society, it was reported that in a randomized, double-blind placebo-controlled trial, pine nut fatty acids increase circulating levels of CCK and GLP-1 concomitant with decreased appetite and increased satiety (Causey J L. Korean pine nut fatty acids induce satiety-producing hormone release in overweight human volunteers. Paper presented at: American Chemical Society National Meeting & Exposition; Mar. 26-30, 2006; Atlanta, Ga.).

It is herein understood by the inventors that the incorporation of a source of pinolenic in a nutritional composition for promoting weight loss will effectively decrease appetite and increase satiety by increasing the circulating levels of CKK and GLP-1.

An embodiment of the present invention comprises between from about 0.001 mg to about 100 mg of Pinolenic acid per serving of the supplemental composition. In an embodiment, the supplemental composition comprises from about 0.01 mg to about 10.0 mg of Pinolenic acid per serving of supplement composition. In a further embodiment, the supplemental composition comprises about 1.0 mg of Pinolenic acid per serving of supplemental composition.

Salacia oblonga

Salacia oblonga extract is known to be an α-glucosidase inhibitor. Glucosidase inhibitors decrease the absorption of carbohydrates from the intestine, resulting in a slower and lower rise in blood sugar following the consumption of a meal. Carbohydrates must be broken down before they can be absorbed from food into simple sugars, such as glucose, by enzymes in the intestine. α-glucosidase is one of the enzymes involved in breaking down carbohydrates. Through the inhibition of this enzyme, carbohydrates are not broken down as efficiently and glucose absorption is thus delayed or at least partially prevented. Heacock et al., 2005. showed that compared to controls in non-diabetic adults a dose of 1000 mg of Salacia oblonga reduced serum glucose and insulin levels by 29% (p=0.01) 120 minutes following the ingestion of a study beverage consisting of 14 g of fat, 82 g of carbohydrates, and 20 g of protein (Heacock P M, Hertzier S R, Williams J A, Wolf B W. Effects of a medical food containing an herbal alpha-glucosidase inhibitor on postprandial glycemia and insulinemia in healthy adults. JAm Diet Assoc. 2005 January; 105(1):65-71). In a separate study, following the administration of a beverage the same as outlined above, it was determined that at 120 minutes following the simultaneous administration of 1000 mg of an extract of Salacia oblonga, plasma glucose was reduced relative to controls by 27% (p=0.035) for the area under the curve measurements. The same study also determined, under the above conditions, that at times of 120 and 180 minutes post-administration of the study beverage and Salacia oblonga extract, there was a 35% and 36% (p<0.001) reduction in serum insulin levels compared to control (Collene A L, Hertzler S R, Williams J A, Wolf B W. Effects of a nutritional supplement containing Salacia oblonga extract and insulinogenic amino acids on postprandial glycemia, insulinemia, and breath hydrogen responses in healthy adults. Nutrition. 2005 July-August; 21(7-8):848-54). These results suggest that the Salacia oblonga extract is effective in decreasing glycemia through its activity as an α-glucosidase inhibitor.

It is herein understood by the inventors that the incorporation of an extract of Salacia oblonga in a nutritional composition for promoting weight loss will effectively inhibit the absorption of carbohydrates from ingested food by acting as an α-glucosidase inhibitor.

An embodiment of the present invention comprises between from about 0.001 g to about 0.1 g of an Extract of Salacia oblonga per serving of the supplemental composition. In an embodiment, the supplemental composition comprises from about 0.005 g to about 0.05 g of an Extract of Salacia oblonga per serving of supplement composition. In a further embodiment, the supplemental composition comprises about 0.01 g of an Extract of Salacia oblonga per serving of supplemental composition.

It is herein understood by the inventors that the components of the present invention, when administered to an individual, will promote weight loss. Said weight loss will be the result of the concomitant action of increased sensations of satiety and decreased absorption of carbohydrates.

Additional embodiments of the present invention may also include portions of the composition as fine-milled ingredients. U.S. Non-Provisional patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling.

For the purposes of the present invention, the terms micronization, milling, particle-milling, and fine-milling are used interchangeably, wherein they refer to a technology, process and end-products involved in or leading to a narrowing of particle size range and a concomitant reduction in the average particle size. For the purposes of the present invention, acceptable milled-particle sizes are in the range of from about 1 nanometer to about 500 microns.

Further to improving bioavailability, it is understood by the inventors that increased solubility resulting from fine-milling will lead to improvements in characteristics in which solubility and reduced particle size likely play a role.

Furthermore, additional embodiments of the present invention may be incorporated into specific controlled-release solid dosage forms. U.S. Non-Provisional patent application Ser. No. 11/709,525 entitled “Method for a Supplemental Dietary Composition Having a Multi-Phase Dissolution Profile” filed Feb. 21, 2007, which is herein fully incorporated by reference, discloses a method of achieving a solid oral dosage form with multiple dissolution characteristics for the release of active ingredients. Conventional oral dosage formulations are bound by the rate of dissolution of the unprocessed substance, thereby limiting the rate of bioavailability of the substance upon oral administration. This is particularly problematic for poorly-soluble compounds which have an inherently low rate of dissolution in that they may be excreted prior to first-pass.

It is herein understood that, due to the relationship between solubility and dissolution, the amount of a substance in solution at any given time is dependent upon both dissolution and solubility. Furthermore, it is understood by way of extension that increasing the rate of dissolution of a given substance acts to reduce the time to dissolution of a given solute or substance in a given solvent. However, the absolute solubility of said solute does not increase with infinite time. Thus, increasing the rate of dissolution of a substance will increase the amount of said substance in solution at earlier points in time, thus increasing the rate of bioavailability of said substance at earlier times upon oral administration.

The increase in the rate of bioavailability will allow better and quicker compound transfer to the systemic parts of the body.

Micronization is a technique which has been used as a method of sizing solid compounds to fine powders. Following a micronization process, compounds and more specifically poorly soluble compounds are transformed into fine powders which can then be transformed into suitable, stable and patient-compliant dosage forms. These forms, for the purposes of the present invention are derived for oral administration.

Micronization techniques offer an advantage over larger forms of compounds and poorly soluble compounds—following micronization, compounds have higher surface area to volume ratio. This provides for, as compared to physically coarse compounds, an ultrafine micronized powder that has a significantly increased total surface area. Mathematically, cross-sectional surface area increases with the square of the radius, while volume increases with the cube of the radius. Therefore, as a particle becomes smaller, the volume of the particle decreases at a faster rate than the surface area leading to an increase in the ratio of surface area to volume. By way of theoretical calculations, decreasing the size of a particle can increase its rate of dissolution via increasing the surface area to volume ratio. In the case of solubility, this increase in relative surface area allows for greater interaction with solvent. Additional embodiments of the present invention may employ a multi-phasic dissolution profile to provide a time-release mechanism.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a caplet or as a liquid capsule.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

The present nutritional composition or those similarly envisioned by one of skill in the art may be utilized in methods to promote weight loss in a formulation designed to be consumed on a daily basis.

Although the following examples illustrate the practice of the present invention in four of its embodiments, the examples should not be construed as limiting the scope of the invention. Other embodiments will be apparent to one of skill in the art from consideration of the specifications and example.

### EXAMPLES

### Example 1

A nutritional supplement is provided in two servings per day as caplets. A single serving of the nutritional composition comprises from about 0.05 g to about 1.5 g of an Extract of Mulberry leaves, from about 0.01 mg to about 10.0 mg of Pinolenic acid and from about 0.005 g to about 0.05 g of an Extract of Salacia oblonga.

Directions: As a diet supplement, 2 caplets are administered with an 8 oz. glass of water two (2) times daily approximately 30 to 60 minutes before meals.

### Example 2

A nutritional supplement is provided in two servings per day as caplets. A single serving of the nutritional composition comprises about 1.2 g of an Extract of Mulberry leaves, about 1.0 mg of Pinolenic acid and about 0.01 g of an Extract of Salacia oblonga.

Directions: As a diet supplement, 2 caplets are administered with an 8 oz. glass of water two (2) times daily approximately 30 to 60 minutes before meals.

Extensions and Alternatives

In the foregoing specification, the invention has been described with a specific embodiment thereof; however, it will be evident that various modifications and changes may be made thereto without departing from the broader spirit and scope of the invention.

What is claimed:

1. A composition for promoting weight loss in an individual comprising:
from about 0.01 g to about 2.0 g of an extract of Mulberry leaf;
from about 0.001 mg to about 100 mg of pinolenic acid; and
from about 0.001 g to about 0.1 g of an extract of Salacia oblonga.

2. The composition of claim 1, wherein the amount of the extract of Mulberry leaf is about 1.2 g;
the amount of the pinolenic acid is about 1.0 mg; and
the amount of the extract of Salacia oblonga is about 0.01 g.

3. The composition of claim 1 wherein at least a portion of one or more ingredients is fine-milled.

4. The composition of claim 1 wherein the extract of Mulberry leaf, the pinolenic acid and the extract of Salacia oblonga comprise an oral dosage form having a multi-phasic rate of dissolution.

5. The composition of claim 4 wherein the multi-phasic rate of dissolution comprises a first-phase and a second-phase; whereby the first-phase has a first rate of dissolution the second-phase has a second rate of dissolution.

6. The composition of claim 5, further comprising a third-phase, whereby the third-phase has a third rate of dissolution.

7. The composition of claim 5, wherein the multi-phasic rate of dissolution provides a time-release mechanism.

8. A method for promoting weight loss in an individual comprising the step of orally administering to the individual a composition comprising:
from about 0.01 g to about 2.0 g of an extract of Mulberry leaf;
from about 0.001 mg to about 100 mg of pinolenic acid; and
from about 0.001 g to about 0.1 g of an extract of Salacia oblonga.

9. The method of claim 8, wherein the composition is administered at least twice daily.

10. The method of claim 9, wherein said composition is administered prior to meals.

11. The method of claim 8, wherein the amount of the extract of Mulberry leaf is about 1.2 g;
the amount of the pinolenic acid is about 1.0 mg; and
the amount of the extract of Salacia oblonga is about 0.01 g.

12. The method of claim 9 wherein at least a portion of one or more ingredients is fine-milled.

13. The method of claim 9 wherein the extract of Mulberry leaf, the pinolenic acid and the extract of Salacia oblonga comprise a solid oral dosage form having a multi-phasic rate of dissolution.

14. The method of claim 14 wherein the multi-phasic rate of dissolution comprises a first-phase and a second-phase; whereby the first-phase has a first rate of dissolution the second-phase has a second rate of dissolution.

15. The method of claim 14, further comprising a third-phase, whereby the third-phase has a third rate of dissolution.

16. The method of claim 14, wherein the multi-phasic rate of dissolution provides a time-release mechanism.
